SEQUENCE LISTING (6 sequences)

SEQ ID NO: 1 - DNA, 40 bp, Artificial Sequence
  Primer
aattaggcgggataattagaattcggcggagagggaatta

SEQ ID NO: 2 - DNA, 30 bp, Artificial Sequence
  Primer
ccctctccgccgaattctaattatcccgcc

SEQ ID NO: 3 - DNA, 18 bp, Artificial Sequence
  Primer
cagcggaggtgccgcttt

SEQ ID NO: 4 - DNA, 89 bp, Artificial Sequence
  Primer
cggcacctccgctgggcgggataactagaactcggcgtgaattcggcaagcttagggatggtagcacttgagacctcgacggcgaccgc

SEQ ID NO: 5 - DNA, 18 bp, Artificial Sequence
  Primer
tttgcggtcgccgtcgag

SEQ ID NO: 6 - DNA, 59 bp, Artificial Sequence
  Primer
tctcaagtgctaccatccctaagcttgccgaattcacgccgagttctagttatcccgcc

(19) United States
(12) Patent Application Publication
(10) Pub. No.: US 2004/0241699 A1
(43) Pub. Date: Dec. 2, 2004

(54) Detection of polynucleotide hybridization

(76) Inventors: Giovanni Zocchi, Los Angeles, CA (US); Mukta Singh Zocchi, Los Angeles, CA (US)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 10/491,353

(22) PCT Filed: Oct. 4, 2002
(86) PCT No.: PCT/US02/31956

(30) Foreign Application Priority Data
Oct. 4, 2001 (US) 60326951

(51) Int. Cl. C12Q 1/68

(52) U.S. Cl. 435/6

(57) ABSTRACT

The invention disclosed herein provides a new detection scheme to monitor hybridization between complimentary polynucleotides such as DNA and/or RNA. Embodiments of the invention disclosed herein localized electromagnetic radiation to provide an optimized analysis of polynucleotide hybridization in contexts such as the polynucleotide microarrays typically used on gene chips.

### RELATED APPLICATIONS

This application claims priority under Section 119(e) from U.S. Provisional Application Serial No. 60/326,951 filed Oct. 4, 2001, the contents of which are incorporated herein by reference.

### FIELD OF THE INVENTION

The present invention provides methods for the detection, identification and/or quantification of polynucleotides such as RNA or DNA and to reagents and detector apparatus adapted for performing these methods.

### BACKGROUND OF THE INVENTION

Gene probe assays, using polynucleotide hybridization, and immunoassays, using immunospecific antibodies, are routinely employed in a wide variety of protocols for the detection and identification of biological materials. Gene probe assays provide a greater versatility than immunoassays in that the hybridization of gene probes for their targets can be controlled to a much greater degree than is possible using protein-based binding phenomena. Moreover, when gene probe assays are coupled with polymerase chain reaction protocols designed to amplify target materials, extreme sensitivity can be obtained.

Polynucleotide (e.g. DNA and RNA) hybridization assays are a central technique in molecular biology, with applications in genomic analysis, gene expression studies, and, increasingly, diagnostics. The sensitivity and scale of the assays have been the subject of continual improvement; in the past few years, DNA arrays were introduced allowing the simultaneous analysis of thousands of hybridization reactions; in addition, several new sensitive detection techniques are being developed. These include molecular beacons (see, e.g. Tyagi et al., Nat. Biotechnol. 14, 303-308 (1996); Tyagi et al., Nat. Biotechnol. 16, 49-53 (1997); Bonnet et al., Proc. Natl. Acad. Sci. USA 96, 6171-76 (1999); and Marras et al., Genet. Anal.-Biomol. E. 14, 151-56 (1999)), nanoparticle composites (see, e.g. Elghanian et al., Science 277, 1078-81 (1997); Storhoff et al., J. Am. Chem. Soc. 120, 1959-64 (1998); Andrew et al., Science 289, 1757-60 (2000); Schultz et al., Proc. Natl. Acad. Sci. USA 97, 996-1001 (2000); and Dubertret, et al., Nat. Biotechnol. 19, 365 (2001)), surface plasmon resonance (SPR) (see, e.g. Peterlinz et al., J. Am. Chem. Soc. 119, 3401-2 (1997); and Heaton et al., PNAS 98, 3701-4 (2001)), fiber optic arrays (see, e.g. Stimpson et al. PNAS 92: 6379-83 (1995); Ferguson et al., Nat. Biotech. 14, 1681-4 (1996); Steemers et al, Nat. Biotechnol. 18, 91-94 (2000); and Yeakley et al., Nat. Biotech. 20, 353-8 (2002)), and conductivity/capacitance measurements (see, e.g. Patolsky et al., Nat. Biotechnol. 19, 253-57 (2001); and So-Jung Park et al., Science 295, 1503 (2002)). The most widely used detection methods rely on labeling the target DNA, most commonly by fluorescent dyes.

DNA arrays (e.g. gene chips) are an important embodiment of gene probe assays in that they permit the measurement of gene expression simultaneously over pools of approximately 104 genes (see, e.g. D. J. Lockhart et al, Nat. Biotechnol. 14, 1675 (1996) and L. Wodicka et al, Nat. Biotechnol. 15, 1359 (1997)). In a typical embodiment of this technology a gene library (the “probe” DNA) is first deposited onto an appropriate matrix in the form of an array (the “gene chip”). Subsequently the sample RNA or DNA, marked with a detectable molecule such as a fluorescent dye, is washed over the chip and allowed to hybridize with the probe. Spots where hybridization occurred are then identified by the resulting fluorescence. Different strategies are employed in preparing the chips, most notably the “in situ synthesis” method of Affymetrix (see, e.g. A. C. Pease et al, PNAS 91, 5022 (1994)), and the “spot spray” method developed by Agilent. The analysis of the hybridized chip is accomplished by a number of means known in the art, for example by a confocal scanner (see, e.g. see, e.g. M. Chee et al, Science 274, 610 (1996) and K. L. Gunderson et al, Genome Res. 8, 1142 (1998).

Unfortunately, a large number of existing hybridization techniques using gene probes are slow, taking from hours to days to produce a result. Biosensors offer an alternative route to fast gene probe assays, but most reports on gene probe biosensor assays are limited to those using surface plasmon resonance (Evans & Charles (1990); Abstracts of 1st World Congress on DNA probes and immunoassay; Pollard-Knight et al (1990) Ann. Biol. Clin, 48 642-646) as well as some preliminary descriptions of methods for carrying out gene probe assays using evanescent wave biosensors, for example by providing a Total Internal Reflection Fluorescence (TIRF) waveguide adapted for carrying out such methods that is incorporated within an evanescent wave biosensor device.

Evanescent wave biosensors, which use the phenomenon of TIRF for detection (Sutherland & Dahne, (1987) J. Immunol. Meth., 74, 253-265), have previously been used with proteins as the biological recognition element. Antibodies have been used to detect the binding of fluorescent-labelled antigen (Eldefrawi et al (1991), Biosensors & Bioelectronics, 6, 507-516) using acetylcholine receptors to study the binding of acetylcholine and cholinesterase inhibitors. Other groups (Poglitsch & Thompson (1990) Biochemistry, 29, 248-254) have measured the binding of antibody to Fc epitopes.

Evanescent wave detectors typically exploit the TIRF phenomenon to provide a sensitive method for detecting reactions at the surface of waveguides. The waveguide can take various forms but typically will be a prism, slab or fiber. The reaction to be used to measure the target molecule can be monitored, for example, through measuring the fluorescence changes on binding or desorption of fluorescent species or by the generation of fluorescent species by enzymatic or chemical means. Several descriptions of the use of evanescent wave detectors in various contexts are known in the art (e.g. U.S. Pat. Nos. 4,582,809, 5,750,337, 5,599,668 and 6,268,125 and U.S. patent application Ser. No. 20020016011) but inherent limitations in existing methods have not allowed the full capabilities of such sensors to be exploited.

Existing polynucleotide microarray technologies are known to exhibit a high level of background noise, a phenomena which can create difficulty in data analysis due to the presence of false positives. This phenomena is due to the fact that RNA or DNA with only short sequence homology to the probe can also hybridize to the probe DNA which produces a signal that is equivalent to those generated by an authentic hybridization signal (where the probe and target sequences have true complementary), thereby confounding the measurement of the authentic signal. Consequently there is a need in the art for additional methods and devices that overcome the host of technical problems that are associated with this technology such as high levels of background noise. The methods and devices disclosed herein satisfy this need.

### SUMMARY OF THE INVENTION

The invention disclosed herein provides new methods and materials for monitoring the hybridization of target polynucleotides to polynucleotide probes having complementary sequences such as those used in polynucleotide microarrays (e.g. gene chips). Preferred embodiments of the invention use localized electromagnetic radiation to provide an enhanced discrimination in the analysis of the signals generated from a polynucleotide microarray. Because such methods alleviate problems associated with high levels of background noise, they have significant advantages over the existing methods in the art. In addition, the invention disclosed herein provides means to efficiently assess both the degree as well as the specificity of polynucleotide hybridization, a feature which will lead to a reduction in the costs of such analytical assays.

Illustrative embodiments of the invention disclosed herein provide methods to detect the annealing or hybridization of a target polynucleotide sequence that is complementary to a polynucleotide sequence in a polynucleotide probe. In a representative embodiment of the invention, a detectable marker such as a fluorescent molecule or light scattering moiety is linked to the free end of a probe polynucleotide, which is preferably DNA. The other end of the probe is coupled (e.g. grafted) to the surface of a matrix such as a chip, with its free end exploring the half space above the surface of the matrix in such a way that the average distance between the detectable marker linked to this free end and the matrix surface depends on the contour length of the probe strand. In this context, the hybridization of a complementary sequence is measured by observing a hybridization induced change in the height of the detectable marker (that is coupled to a polynucleotide probe's free end) above the surface of the chip.

In typical methods, a signal generated by polynucleotide hybridization is correlated to a measure of the average height of the marker coupled to a polynucleotide probe's free end (e.g. a fluorophore) above the surface of the chip. For example, in certain embodiments, upon hybridization with a complementary polynucleotide sequence, the probe shortens, which changes the contour of the probe and hence the height of the detectable marker above the matrix to which the probe is coupled. This hybridization modulated change in the height of the detectable marker above the matrix can then be measured by methods known in the art. Preferably the hybridization is measured via evanescent wave illumination.

In a specific illustrative example using a fluorescent labelled DNA probe, exciting with the 488 nm line of an Ar laser, the penetration depth of the evanescent wave is 50 nm, which translates into a ˜2% increase in a fluorescent signal for every 1 nm change in the fluorophore's average vertical position. A probe consisting of a sequence 60 bases long can then lead to a ˜15% change in fluorescent or scattered intensity for complete annealing. Consequently, when a complementary polynucleotide hybridizes to a probe sequence, this contour length, and thus the average fluorescent-surface distance is reduced, which causes a subsequent increase in the fluorescent signal. This annealing modulated change in the fluorescent signal can then be measured by one of the methods known in the art, for example by detection with evanescent wave illumination.

The disclosure provided herein further demonstrates the extreme sensitivity of the methods of the invention, for example the detection of nm scale conformational changes of single DNA oligomers through a micro-mechanical technique. In these methods, the quantity monitored is the displacement of a μm size bead tethered to a surface by the probe molecule undergoing the conformational change. This technique allows to probe conformational changes within distances beyond the useful range of Fluorescence Resonance Energy Transfer (FRET). For example, one can apply the method to detect single hybridization events of label-free target oligomers. As noted above, hybridization of the target is detected through the conformational change of the probe.

The methods disclosed herein have a number of embodiments. A typical embodiment of the invention is a method of detecting hybridization between a polynucleotide probe and a target polynucleotide having a nucleic acid sequence that is complementary to a nucleic acid sequence in the polynucleotide probe, wherein a first end of the polynucleotide probe is coupled to a matrix and a second end of the polynucleotide probe is coupled to a detectable marker, the method including observing a change in the conformation of the polynucleotide probe that is the result of hybridization between the polynucleotide probe and the target polynucleotide. In preferred embodiments, the change in the conformation of the polynucleotide probe is observed by observing a decrease in the height of the detectable marker above the surface of the matrix that results from the hybridization between the polynucleotide probe and the target polynucleotide. In alternative embodiments, the change in the conformation of the polynucleotide probe is observed by observing an increase in the height of the detectable marker above the surface of the matrix that results from a stiffening of the probe that is the result of hybridization between the polynucleotide probe and the target polynucleotide. In highly preferred embodiments of the invention, the change in the conformation of the polynucleotide probe (e.g. the change in the height of the detectable marker above the surface of the matrix) is observed using evanescent wave scattering.

As disclosed herein, the methods of the invention allow the examination of different aspects of hybridization between a polynucleotide probe and a target polynucleotide having a nucleic acid sequence that is complementary to a nucleic acid sequence in the polynucleotide probe. In preferred embodiments for example, a hybridization induced change in the conformation of the probe is correlated to the degree of complementarity between the probe and the target polynucleotide. In yet another embodiment, the hybridization induced change in the conformation is correlated to the relative amounts of the polynucleotide probe and the target polynucleotide.

A variety of alternative embodiments of the methods of the invention are disclosed herein. In one such embodiment, the target polynucleotide is also labelled with a detectable marker. Alternatively, the target polynucleotide is not labelled with a detectable marker. In addition, in preferred methods of the invention, the polynucleotide probe is about 10 to about 400 nucleotide residues in length, preferably about 20 to about 300 nucleotide residues in length, and more preferably about 30 to about 200 nucleotide residues in length. In typical embodiments, the matrix is a gene chip including a plurality of polynucleotide probes. Moreover, the detectable marker is typically a fluorescent compound, a polymer bead or a light scattering particle. Highly preferred methods of the invention include creating a negative charge on the surface of the matrix, which can be accomplished for example by immobilizing negatively charged molecules on the surface of the matrix.

Yet another embodiment of the invention is a method of detecting hybridization between a polynucleotide probe and a target polynucleotide having a nucleic acid sequence that is complementary to a nucleic acid sequence in the polynucleotide probe, wherein the polynucleotide probe has a first end labeled with a detectable marker and a second end attached to a matrix having a negative charge, the method including using evanescent wave illumination to observe a reduction in the height of a detectable marker coupled to the polynucleotide probe's free end above the surface of the matrix to which the polynucleotide probe is attached. In highly preferred embodiments, the detectable marker is a fluorescent compound or a light scattering particle. Optionally, the target polynucleotide is not labelled with a detectable marker and/or the matrix is a gene chip includes a plurality of polynucleotide probes.

Yet another embodiment of the invention is a method of detecting hybridization between a polynucleotide probe and a target polynucleotide having a nucleic acid sequence that is complementary to a nucleic acid sequence in the polynucleotide probe, wherein the polynucleotide probe has a bound end coupled to a matrix and a free end coupled to a detectable marker, the method including determining an height of the detectable marker coupled to the polynucleotide probe's free end above the surface of the matrix to which the probe is attached in the absence of a complementary polynucleotide sequence, allowing the polynucleotide probe and the target polynucleotide sequence to come into contact with one another under conditions favorable to hybridization, using evanescent wave illumination to measure the height of the detectable marker coupled to the polynucleotide probe's free end above the surface of the matrix to which the probe is attached in the presence of the target polynucleotide sequence; comparing the height of the detectable marker in the absence of complementary polynucleotide sequences with the height of the detectable marker in the presence of target polynucleotide sequences, wherein a reduction the height of the detectable marker in the presence of target polynucleotide sequences is indicative of hybridization between a polynucleotide probe and a target polynucleotide having a nucleic acid sequence that is complementary to a nucleic acid sequence in the polynucleotide probe.

Yet another embodiment of the invention is an apparatus for detecting hybridization between a polynucleotide probe and a target polynucleotide having a nucleic acid sequence that is complementary to a nucleic acid sequence in the polynucleotide probe, wherein the hybridization is detected using evanescent wave illumination, the apparatus including a matrix on which a first end of a polynucleotide probe attached, wherein the second end of the polynucleotide probe is coupled to a detectable marker consisting of a fluorophore or a light scattering marker; a coupling mechanism which optically couples the probe to an optical guide to obtain an evanescent wave on the surface of the matrix; an optical arrangement which measures the fluorescent or scattered intensity both before and after depositing a solution containing a target polynucleotide sequences on the probe under conditions which favor hybridization of the probe and a target polynucleotide sequences that are complementary to a nucleic acid sequence in the polynucleotide probe; and a detector which records the difference of fluorescent intensity or scattering before and after subjecting the probe DNA to the target polynucleotide sequences.

The invention also provides articles of manufacture and kits which include one or more elements used in performing the methods of the invention and instructions for their use. Another preferred embodiment of the invention is a kit including a container, a label on said container, and a polynucleotide probe composition contained within said container; wherein a first end of the polynucleotide probe is coupled to a matrix and a second end of the polynucleotide probe is coupled to a detectable marker; and instructions for using the polynucleotide probe composition in methods of detecting hybridization between a polynucleotide probe and a target polynucleotide having a nucleic acid sequence that is complementary to a nucleic acid sequence in the polynucleotide probe by observing a change in the conformation of the polynucleotide probe that is the result of hybridization between the polynucleotide probe and the target polynucleotide. In preferred embodiments of the kits, the detectable marker is selected to be compatible for use with evanescent wave illumination. In highly preferred embodiments, the matrix is a gene chip having a negatively charged surface.

### BRIEF DESCRIPTION OF THE FIGURES

FIG. 1. Illustration of how the vertical position of the bead changes as a consequence of inducing an elongation of the tethering DNA. This conformational change is induced by introducing in the flow cell an intercalating agent (Ethidium Bromide), which is known to produce an elongation of ds DNA of about 30%. The figure shows the vertical position of the bead (h, in nm) in the course of time. Because the bead is tethered by several DNA molecules, its thermal motion is suppressed to an extent that one can measure its vertical position with sub nm resolution, as is apparent from the figure. Between t=70 s and t=100 s the solution surrounding the bead (phosphate buffered 25 mM NaCl solution) is slowly exchanged with the same solution containing Ethidium Bromide. As the tethers elongate, the bead moves approximately 3 nm further away from the microscope slide; this is the expected magnitude of the effect, because the initial length of the 30 bp oligomer is approximately 10 nm, so a 30% elongation would correspond to a 3 nm displacement. This measurement shows that the sensitivity of the method is appropriate for the intended purposes.

FIG. 2. Illustration of the limit of a single molecular tether: here the surface concentration of binding sites (Avidin) on the slide was sufficiently low that on average a bound bead will be tethered by only one oligo. The thermal motion of the bead (which is mainly a pivoting motion around the tethered point) is now much bigger, with an amplitude of roughly 10 nm; the figure also shows the effect of a flow on the bead under these conditions: at times 10<t<22 s , 30<t<45 s , 60<t<63 s a flow on in the cell, which pushes the bead down against the bottom.

FIG. 3. Results from a control experiment illustrating how the beads are specifically bound by DNA tethers. Specifically, by introducing DNase, the tethers are cut and the bead is eventually released, as can be seen by the increase in amplitude of the Brownian motion.

FIG. 4. Results from a hybridization experiment in which the bead is tethered by a more complicated construct: a 60 bases long DNA oligonucleotide, which is partly (30 bases) double stranded and partly (30 bases) single stranded. When a polynucleotide complementary to the single stranded sequence is introduced, a downward shift of the bead is observed which corresponds to a contraction of the tethers, in this case by about 2 nm.

FIGS. 5A and 5B. (A) The two schemes used to tether 1 μm diameter beads through a probe oligomer. (B) The upper part of the Figure shows schematically the optical setup; the lower part shows the principle of the measurement

FIG. 6. Relative bead-surface separation h, in nm, measured in the course of time by evanescent wave scattering. The bead is tethered by the 40 mer C40 (SEQ ID NO: 1); a single hybridization event with a complementary 30 mer (C40*, SEQ ID NO: 2) pulls the bead ˜2 nm closer to the surface. Target concentration was 500 nM. The absolute h is not measured directly; it corresponds to an average value of the contact intensity Ic determined separately.

FIGS. 7A and 7B. (A) A bead tethered by the 90 mer C90 (SEQ ID NO: 4) shows large (˜6 nm) vertical thermal fluctuations. A horizontal flow pushes the bead closer to the surface (4<t<6 min and 14<t<16). Approximately 20 min after a complementary 60 mer is introduced (at a concentration of 20 nM), a single hybridization event (t≈40 min) pulls the bead towards the surface by ˜5 nm; the amplitude of the vertical fluctuations is also reduced. (B) Signature of a single hybridization event obtained with a target concentration of 2 nM. This is a different bead and cell, but conditions are otherwise the same as in FIG. 7A.

FIG. 8. The case of many (C90) tethers. Vertical fluctuations are smaller, but a flow still has a visible effect (5<t<7 and 17<t<19). Upon hybridization the tethers stiffen, pushing the bead away from the surface (t≈22).

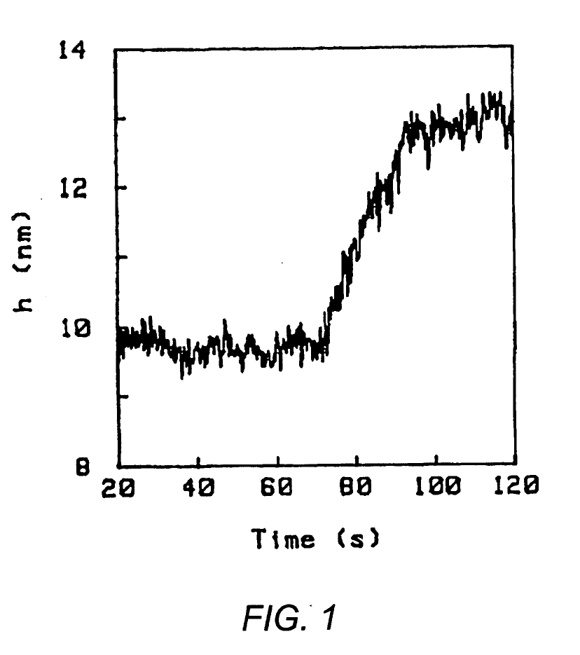

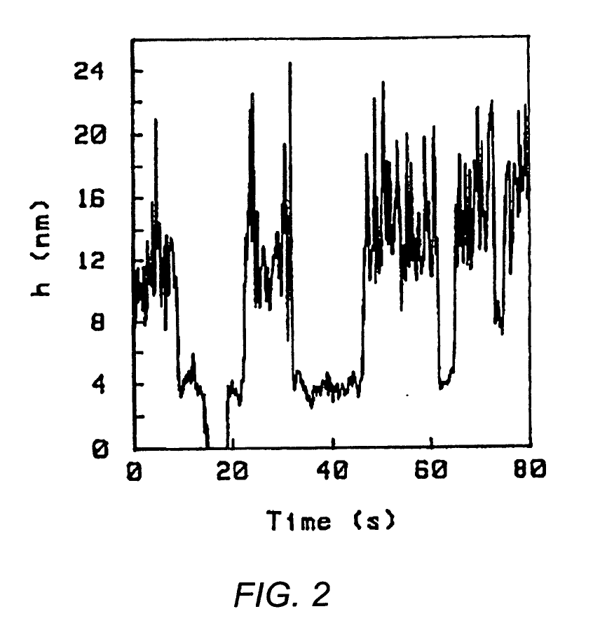

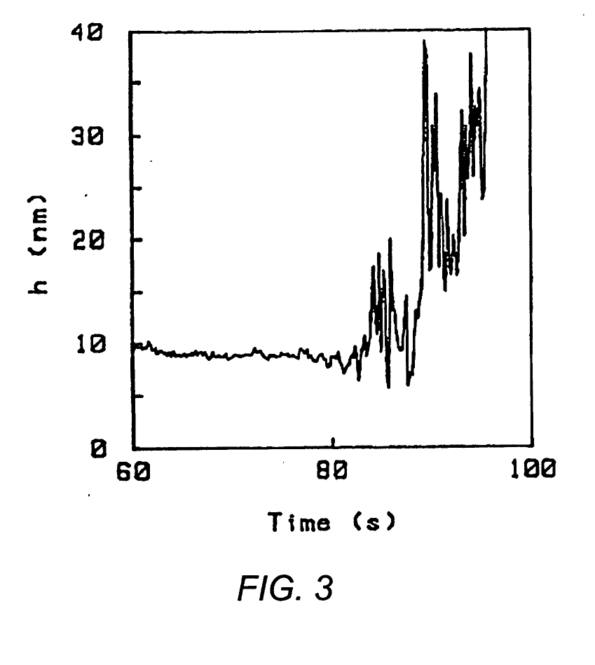

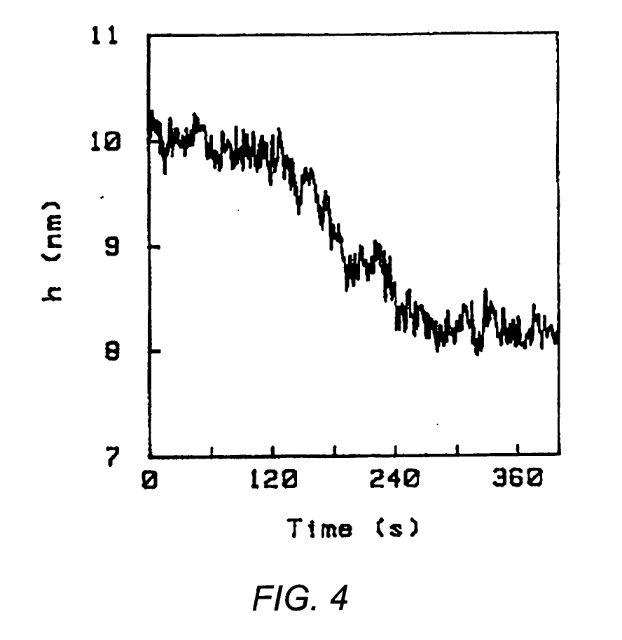

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Embodiments of the invention are directed to methods of detecting hybridization between a polynucleotide probe and a target polynucleotide having a nucleic acid sequence that is complementary to a nucleic acid sequence in the polynucleotide probe. As noted above, unless otherwise indicated the terminology used in the description of these embodiments are intended to have the meanings commonly understood by those of skill in the art to which this invention pertains (see, e.g. Oxford Dictionary of Biochemistry and Molecular Biology (1997) Oxford University Press A. D. Smith Managing Editor). In this context, the term “polynucleotide” means a polymeric form of nucleotides of at least about 10 bases or base pairs in length, either ribonucleotides or deoxynucleotides or a modified form of either type of nucleotide, and is meant to include single and double stranded forms of DNA and/or RNA. As is known in the art, such polynucleotides typically have two termini, a 3′ and a 5′ end. In the methods of the invention, a first end of the polynucleotide probe is coupled to a matrix such as the surface of a gene chip and a second end of the polynucleotide probe is coupled to a detectable marker. As used herein, a “detectable marker” simply refers to one of the various agents that artisans couple to polynucleotide sequences in order to facilitate their detection (e.g. via evanescent wave illumination as disclosed herein). Preferred detectable markers include fluorophores as well as light scattering moieties which include for example, small metal particles, polymer beads and the like.

The methods of the invention comprise observing a change in the conformation of the polynucleotide probe that is the result of hybridization between the polynucleotide probe and the target polynucleotide. As used herein, the terms “hybridize”, “hybridizing”, “hybridizes” and the like, used in the context of polynucleotides, refers to the process wherein complementary single stranded polynucleotides (e.g. DNA and/or RNA) form duplex molecules upon being annealed together. “Complementary” as in a complementary base pair sequence refers to a sequence in a polynucleotide chain that is able to form base pairs with a sequence of bases in another polynucleotide chain.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, are exemplified by: (1) hybridization in 50% formamide, 2×SSC, 0.1% SDS, 10 mg/ml salmon sperm DNA, and 10% dextran sulfate, at 42° C. for 16 hours followed by a washing in 2×SSC, 0.1% SDS at 25° C. for 10 min (three times), and washed in the same solution at 65° C. for 5 min (twice) and are generally identified by, but not limited to, those that: (2) employ conditions of low ionic strength and high temperature for washing, for example 0.015 M sodium chloride/0.0015 M sodium citrate/0.1% sodium dodecyl sulfate at 50° C.; (3) employ during hybridization a denaturing agent, such as formamide, for example, about 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (4) employ 50% formamide, about 2-5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

The invention disclosed herein provides a new detection scheme to monitor annealing of target polynucleotides such as DNA and/or RNA on a matrix such as a polynucleotide microarray such as those typically used on gene chips. Typical methods described herein use localized electromagnetic radiation to provide an enhanced discrimination in the analysis of these polynucleotide microarray. Because the methods are versatile, and for example, are not restricted to the use of fluorescent markers, they provide means for more cost-effective devices. Consequently, the invention described herein provides a new method for use in the variety of microarray technologies known in the art. As illustrated below, the invention alleviates problem associated with high levels of background noise and will lead to reduced costs and better specificity for hybridization.

The invention disclosed herein provides methods and materials to detect polynucleotide hybridization through a hybridization induced conformational change in the polynucleotide probe. Such methods have advantages over existing methods by, for example, eliminating the need to label the target. Here we demonstrate a micro-mechanical method, which exploits a conformational change in a single probe molecule to detect hybridization of a single target. In our experiment, we detect the shortening of the contour length of the probe oligomer caused by the formation of the double helix upon hybridization. In a variant of the experiment we detect instead the stiffening of the probe oligomers caused by hybridization. The detection limit of the method is in principle a single target molecule. Here we report detection of a specific unlabelled target sequence at a concentration of 2 nM, in a total volume of 80 μl, and in the presence of 50 fold excess concentration of unrelated oligomers.

In an illustrative embodiment of the invention, micron size polystyrene beads are tethered to the surface of a microscope slide by a single DNA oligonucleotide (the probe), of length 40-90 bases. The bead is prevented from sticking to the slide by a repulsive electrostatic barrier due to surface charges; at the same time it cannot break loose from the slide because of the molecular tether (see, e.g. Zocchi et al., Biophys. J. 81, 2946-53 (2001)). Hybridization of the target to the probe shortens the molecular tether, pulling the bead closer to the slide. The bead-slide separation is monitored with sub-nm resolution by evanescent wave scattering (see, e.g. Zocchi et al., Biophys. J. 81, 2946-53 (2001); and Singh-Zocchi et al., PNAS 96, 6711-15 (1999)).

A variant of the experiment is the opposite limit of a bead held by many tethers, i.e. heavily constrained. In this case, upon hybridization the bead is pushed away from the surface; the origin of this effect is the stiffening of the tethers.

The experimental results provided herein demonstrate the label free detection of single hybridization events. Because the signal is inherently independent of target concentration and amount, very low detection limits seem possible with this method.

Different methods employing the use of evanescent waves to detect hybridization have been proposed before (see, e.g. U.S. Pat. No. 5,750,337). Such methods however, are not related to gene chip technology, and do not employ methods in which the probe DNA is marked, but instead describe methods wherein RNA is marked, methods which involve significantly different technical protocols from those described herein. In contrast, the current invention, which discloses methods involving the marking of a chip, provide significant advantageous features, for example the use of the same marked molecules in multiple hybridization procedures. Typical embodiments of the invention are provided below.

In a generalized illustrative embodiment, a probe polynucleotide such as a DNA is end-grafted on to an appropriate matrix such as the solid surface of the chip (typically made of one of the preferred materials in this art such as glass, quartz, mica, etc.), using one of the variety of techniques typically used in the art, for example amino linkers, biotin-avidin, or thiol chemistry. The opposite (free) end of the probe DNA is marked with a fluorophore or with an attached scatterer (which can be, for example, a nanometer size gold particle or a submicron size polymer bead or another such scatterer known in the art). In this context, a variety of fluorophore detectable markers are also known in the art (see, e.g. U.S. Pat. No. 6,440,705). In addition, a variety particles that reflect or scatter light are known in the art as signal responsive moieties. A light reflecting and/or scattering particle is typically a molecule or a material that causes incident light to be reflected or scattered elastically, i.e., substantially without absorbing the light energy. Such light reflecting and/or scattering particles include, for example, metal particles, colloidal metal such as colloidal gold, colloidal non-metal labels such as colloidal selenium, dyed plastic particles made of latex, polystyrene, polymethylacrylate, polycarbonate or similar materials (see, e.g. U.S. Pat. No. 6,342,349).

Embodiments of the invention disclosed herein are based on detecting the fluorescent intensity of the probe in an evanescent wave setup; this intensity is a measure of the average height of a detectable marker such as a fluorophore that is coupled to the probe's free end above the surface of the chip. Specifically, upon hybridization with the complementary RNA or DNA the probe shortens, giving rise to an increase in the fluorescent signal For example, exciting with the 488 nm line of an Ar laser, the penetration depth of the evanescent wave is 50 nm, which translates into a ˜2% increase in fluorescent signal for every 1 nm change in the fluorophore's average vertical position. Consequently, a probe consisting of a sequence 60 bases long could then lead to a ˜15% change in fluorescent or scattered intensity for complete annealing. Moreover, the change in fluorescent signal is a measure of the degree of hybridization, a change which can easily be detected.

Under conditions where the probe DNA is saturated by the target RNA or DNA (excess of target), the present method measures, for each probe, the degree of annealing, and can thus distinguish the signal generated by true complementaries from the signal generated by spurious partial homologies. Under conditions where the probe DNA is not saturated (excess of probe) one can measure both the degree of annealing and the amount annealed with the present method, thus distinguishing a true complementary and measuring its amount present For this purpose, the target DNA can also be marked fluorescently, with a dye different from the probe's (which, alternatively, could be marked with a scatterer). From the two measurements, amount of fluorescence due to the target and change in probe's fluorescence or scattering intensity one extracts the information mentioned above.

A specific illustrative embodiment of the invention entails the following steps. In a first step, one obtains a chip, of the approximate size of a microscope slide, made of glass, or quartz, or mica covered quartz, or similar transparent material where the probe DNA, typically 30-300 bases in length, is attached by one end, through an amino linker, biotin-avidin complex, Dig-anti DIG complex, thiol group, or similar chemistry. The free end of the probe DNA is tagged with a fluorescent dye, or alternatively with a small (micron to sub micron size) scatterer, e.g. a polymer bead, colloidal gold particle, etc. A second step entails coupling this chip through an index matching fluid to a prism or similar waveguide for the purpose of steering a light beam in such a way to obtain an evanescent wave at the surface of the chip. A third step entails obtaining a measurement of the fluorescent or scattered intensity for all the spots in the array, using a microscope objective and CCD camera to collect the light, or an objective and photomultiplier tube and scanning across the chip, or similar light detection scheme. A fourth step entails washing the solution containing the target RNA or DNA, which may or may not be itself fluorescently tagged (as mentioned above), on the chip under conditions that favor annealing to the probe. A fifth step entails obtaining a second measurement of the fluorescent or scattered intensity for all the spots in the array; the difference with the measurement in the third step reflects the degree of annealing of the target to the probe. In the case where the target RNA or DNA was fluorescently labeled, obtaining a measurement of the corresponding fluorescent intensity for all the spots in the array; from these data and the data obtained in the fifth step one calculates both the degree of annealing and the amount of target RNA or DNA present on the chip, for all spots.

As noted above, the invention disclosed herein has a number of embodiments. In one embodiment of the present invention, a fluorescent molecule is linked to the free end of the probe DNA. This can be obtained, for example, as the last step of the “in situ” synthesis method developed by Affymetrix, or with any of the standard linking methods (see, e.g. Molecular Probes). The other end of the probe DNA being grafted to the surface of the chip, it will be advantageous to maintain a negative charge on this surface, both to minimize non specific sticking of the target RNA or DNA and to ensure that the probe DNA stands off from the surface, its free end exploring the half space above the surface in such a way that the average distance between the fluorophore linked to this free end and the surface depends on the contour length of the DNA strand. When the target RNA or DNA hybridizes, this contour length, and thus the average fluorophore-surface distance, is reduced. This decrease in the average fluorophore-surface distance then causes an increase in the fluorescent signal. This increase in the fluorescent signal can then be measured by methods known in the art, for example with evanescent wave illumination.

An average negative charge can be maintained on the surface of the chip by immobilizing negatively charged molecules on the surface. Thus, apart from the end grafted probe DNA, the surface of the chip can be covered by a molecular layer, for example a protein monolayer, the measurements being then performed at a pH such that this layer is negatively charged.

In another embodiment of the present invention, a scatterer is linked to the free end of the probe DNA. The scatterer can be any particle of appropriate size, from micrometer to nanometer size, with an index of refraction which provides sufficient contrast with respect to the surrounding solvent. Examples are polymer beads and colloidal gold particles. The particle can be linked to the end of the probe by a variety of methods, for example an amino-derivatized bead can be covalently linked to the amino-modified probe DNA, the probe DNA can be biotinylated at the end and linked to a streptavidin derivatized bead, and so on. The beads can be tethered by a single probe molecule each, or by several; likewise, one can have a single bead per spot on the array, or several. The measured quantity is now the intensity of the light scattered by the beads, with evanescent wave illumination. The beads are tethered by the probe DNA; upon hybridization with the target, the contour length (and the rigidity) of the tether changes, which is reflected in a shift in the average position of the bead above the surface of the chip; this is detected as a change in intensity of the scattered light.

Another variation of the invention disclosed herein utilizes a 1 micron size polystyrene bead and a 10 nm size colloidal gold particle, examples which represent two members of the wide spectrum of detectable markers that can be employed in the methods disclosed herein. With a 1 micron size polystyrene bead, even for a single bead the scattered intensity is very strong compared to the background, and one can easily measure the average intensity to better than 1%, and correspondingly the average “vertical” position of the bead within a fraction of 1 nm. The measurement can be performed on a single bead, which entails the possibility of having only a minute amount of probe DNA per spot, the realistic limit being in fact a single probe DNA molecule per spot. This can translate into an extreme sensitivity to minute amounts of target DNA. However, a better strategy can be to bind the bead through several DNA tethers, but with the actual number of molecules still being small. In this case also it will be advantageous to control the surface charge on the chip and the beads; in fact the bead-surface interaction potential can easily be tuned, by controlling surface charge and ionic strength. In this configuration it is therefore possible to use the bead to gently stretch the probe DNA away from the surface of the chip, which is the preferred configuration for our measurement.

In the case of very small scatterers such as 10 nm size colloidal gold particles, the scattered intensity is at best comparable to the background for a single scatterer. For single scatterers, the detection sensitivity required is comparable to the requirements for single molecule fluorescent detection. The preferred method will then be to use many scatterers per spot on the array, each typically tethered by one probe DNA molecule. Also, in this case the bead-surface long range interaction is weak.

The scattering methods have, in principle, several advantages over fluorescent methods. For example, there is no bleaching of the fluorophore, so measurements can be averaged for long times and the chip is, from this point of view, completely reusable. In addition, a large (micron size) scatterer entails the possibility of obtaining great sensitivity, perhaps down to single molecule sensitivity, because one can work with very small amounts of probe DNA; the signal (the scattered intensity) is still the same.

In addition to providing a novel type of detection scheme for hybridization, the general techniques disclosed herein offer additional important advantages. For example, the fluorescent dye or scatterer can be coupled to a reusable probe, which makes the system less costly and more efficient. Moreover, using the methods disclosed herein, one can measure the specific degree of annealing as a function of the change in probe shortening (and thus the change of the evanescent wave signal) which is proportional to the hybridized fraction. Therefore the method provides significant advantages by distinguishing false positives from authentic signals, leading to lower background and greater sensitivities of polynucleotide detection.

The invention disclosed herein has a number of embodiments. One embodiment is a method of using evanescent wave excitation or a combination of evanescent wave and transmission excitation (e.g. in a confocal geometry) to measure the amount of a DNA probe annealed to a target polynucleotide sequence and the degree of the DNA probe annealed to the target polynucleotide sequence. Another embodiment is a method of detecting the hybridization of a polynucleotide probe to a complementary polynucleotide sequence which involves labeling the polynucleotide probe with a fluorophore and detecting a hybridization induced change in the fluorescence signal in response to evanescent wave excitation. Another embodiment is a method of detecting the hybridization of a polynucleotide probe to a complementary polynucleotide sequence which involves labeling the polynucleotide probe with a scatterer and measuring the scattering of an evanescent wave.

Yet another embodiment of the invention is a method of detecting the hybridization of a polynucleotide probe to a complementary polynucleotide sequence wherein the polynucleotide probe has a free end coupled to a detectable marker and an end attached to a matrix, the method comprising measuring the average height of a marker coupled to the polynucleotide probe's free end above the surface of the matrix to which the probe is attached, wherein the measure of the average height of the marker above the surface of the matrix is correlated to a degree of complementarity between the polynucleotide probe and the complementary polynucleotide sequence or to the amount of complementary polynucleotide sequence that is hybridized to the polynucleotide probe. Preferably in these methods the average height of the marker coupled to the polynucleotide probe's free end above the surface of a matrix to which the probe is attached is measured via evanescent wave illumination.

Yet another embodiment of the invention is a method of using evanescent wave illumination to detect a hybridization between a polynucleotide probe and a target polynucleotide sequence that is complementary to the polynucleotide probe, wherein the polynucleotide probe has a bound end coupled to a matrix and a free end coupled to a detectable market, the method comprising: measuring an average height of the marker coupled to the polynucleotide probe's free end above the surface of the matrix to which the probe is attached in the absence of the target polynucleotide sequence; allowing the polynucleotide probe and the target polynucleotide sequence to come into contact with one another under conditions favorable to hybridization; measuring the average height of the marker coupled to the polynucleotide probe's free end above the surface of the matrix to which the probe is attached in the presence of the target polynucleotide sequence; comparing the measurement value obtained in the absence of target polynucleotide with the measurement value obtained in the presence of target polynucleotide; wherein the measure of the average height of the marker above the surface of the matrix is correlated to factor selected from the group consisting of a degree of complementarity between the polynucleotide probe and the target polynucleotide sequence and the amount of target polynucleotide sequence hybridized to the polynucleotide probe.

Yet another embodiment of the invention is a method of using evanescent wave illumination to determine the degree of complementarity between a polynucleotide probe and a polynucleotide sequence complementary to the polynucleotide probe, wherein the polynucleotide probe has a free end and an end attached to a matrix, the method comprising measuring the average height of a marker coupled to the polynucleotide probe's free end above the surface of the matrix to which the probe is attached, wherein the measure of the average height of the marker above the surface of the matrix is correlated to a degree of complementarity between the polynucleotide probe and the polynucleotide sequence complementary to the polynucleotide probe and wherein the average height of the marker above the surface of the matrix is measured using evanescent wave illumination.

A preferred embodiment of the invention is a method of using evanescent wave illumination to detect the annealing between a plurality of polynucleotide probes and one or more complementary polynucleotide sequences wherein the polynucleotide probe has a free end to which is attached a detectable marker and an end attached to a matrix, the method comprising measuring the average height of a marker coupled to the polynucleotide probe's free end above the surface of the matrix to which the probe is attached, wherein the average height of the marker above the surface of the matrix is correlated to the presence of complementary polynucleotide sequences as well as a degree of complementarity between the polynucleotide probe and the complementary polynucleotide sequence. Alternatively, the average height of the marker above the surface of the matrix is correlated to the relative amount of complementary polynucleotide sequences that are annealed to the polynucleotide probes.

Yet another embodiment of the invention is a method of using evanescent wave illumination to detect annealing between a polynucleotide probe and a target polynucleotide sequence that is complementary to the polynucleotide probe, wherein the polynucleotide probe has a bound end coupled to a matrix and a free end coupled to a marker, the method comprising: measuring an average height of the marker coupled to the polynucleotide probe's free end above the surface of the matrix to which the probe is attached in the absence of the target polynucleotide sequence; allowing the polynucleotide probe and the target polynucleotide sequence to come into contact with one another under conditions favorable to annealing; measuring the average height of the marker coupled to the polynucleotide probe's free end above the surface of the matrix to which the probe is attached in the presence of the target polynucleotide sequence; comparing the measurement value obtained in the absence of target polynucleotide with the measurement value obtained in the presence of target polynucleotide; wherein the measure of the average height of the marker above the surface of the matrix is correlated to factor selected from the group consisting of a degree of complementarity between the polynucleotide probe and the target polynucleotide sequence and the amount of target polynucleotide sequence hybridized to the polynucleotide probe.

The methods presented herein can be used to detect a nm scale conformational change of a single 10-30 nm long DNA oligonucleotide, and we have applied the technique to the detection of a single hybridization event. Mechanical manipulations of single DNA molecules have been performed previously, but at larger scales (λ-DNA, ˜15 μm long) (see, e.g. Cluzel et al., Science 271, 792-4 (1996); Smith et al., Science 271, 795-99 (1996); and Strick et al., Nature 404, 901-4 (2000)). Nanometer scale conformational changes of single molecules have been observed by fluorescence energy transfer (FRET) (see, e.g. Zhuang et al., Science 288, 2048-51 (2000)), and atomic force microscopy (AFM) (see, e.g. Radmacher et al., Science 265, 1577-79 (1994)). However, the method described here can detect conformational motion between parts of a molecule which are beyond the useful range for FRET (>10 nm); this is the case for the end-to-end distance of our ˜20 nm long oligomers. Compared to the AFM, the method has the advantage of technical ease and scalability.

In such embodiments, the size of our probe (typically 40-90 bases) is adapted to hybridization studies; because single hybridization events are detected, the method holds the promise of a very low detection limit in terms of total amount of target The invention disclosed herein therefore has applications in the gene expression analysis of small subpopulations of cells, such as are encountered in stem cell research. Optionally the methods can be used to perform such analysis on single cells, in order to explore cell to cell variations.

Further embodiments of the invention will include moving from detection alone to measuring the amount of target. These embodiments involve collecting the signal from many, smaller beads. Other embodiments include optimized (e.g. covalent) attachment of the probe oligomers to the surfaces, optimized surface chemistry to minimize non specific sticking of the beads, and the control of bead-slide interactions and hybridization rates through an electric field (see, e.g. Heaton et al., PNAS 98, 3701-4 (2001)). Finally, this system can be used to directly detect other kinds of conformational changes in DNA oligomers, such as those induced by protein binding.

Embodiments of the invention also include apparatus designed to carry out the methods of the invention. A typical embodiment is an apparatus for detecting the fluorescence or scattering of evanescent wave, the apparatus comprising: a substrate on which a probe DNA, or an array of DNA probes is deposited; means for tagging the probe with fluorescent dye or a micron or submicron sized scatterer; a coupling mechanism which optically couples the probe to an optical guide to obtain an evanescent wave on the surface of a chip; an optical arrangement which measures the fluorescent or scattered intensity both before and after depositing a solution containing a target RNA or DNA on the probe under conditions which favor annealing of the probe; and a detector which records the difference of fluorescent intensity or scattering before and after subjecting the probe DNA to the target RNA or DNA.

Embodiments of the invention also include kits designed to facilitate the methods of the invention. For use in the applications described or suggested above, kits are also provided by the invention. Typically such kits include instructions for using the elements therein according to the methods of the present invention. Such kits can comprise a carrier means being compartmentalized to receive in close confinement one or more container means such as vials, tubes, and the like, each of the container means comprising one of the separate elements to be used in the method. For example, one of the container means can comprise a probe (a probe attached to a gene chip for example) that is or can be detectably labeled with a marker as described above. Such probe can be a polynucleotide specific for a specific gene or message, respectively. As the kit utilizes nucleic acid hybridization to detect the target nucleic acid, the kit can also have containers containing buffers for the hybridization of the target nucleic acid sequence and/or a container comprising a reporter-means, such as a fluorophore or scattering molecule.

Throughout this application, various publications are referenced. The disclosures of these publications are hereby incorporated by reference herein in their entireties.

### EXAMPLES

### Example 1

### Illustrative Materials and Methods

A. Typical Flow Cells.

Flow cells were constructed with a microscope slide and cover glass separated by 75 μm thick spacers and glued together; typical cell volume was 80 μL. Slides were previously washed with soap and water in an ultrasound bath, rinsed, cleaned with “piranha solution” (5 parts water, 1 part H₂O₂, 1 part H₂SO₄) at 60° C. for 15 min, rinsed, silanized with AquaSil (Pierce) for 15 min, rinsed, baked for at least 30 min at 100 C. Some experiments were also performed with non-silanized slides.

B. Typical Preparation of Tethered Beads.

All DNA oligonucleotides were purchased from Operon Inc., HPLC purified. The experiments were performed on beads tethered to the bottom of the flow cell (formed by the upper surface of the slide). In one scheme (1), the probe was a 40 mer (C40) modified with digoxigenin (DIG) at one end and biotin at the other end. Amino-modified, 1 μm diameter polystyrene beads (Polysciences) were functionalized with anti-DIG by incubating in a 8% solution of gluteraldehyde (in PBS) followed by coupling of anti-DIG (Fab fragment, Roche), blocking by BSA, and coupling to C40.

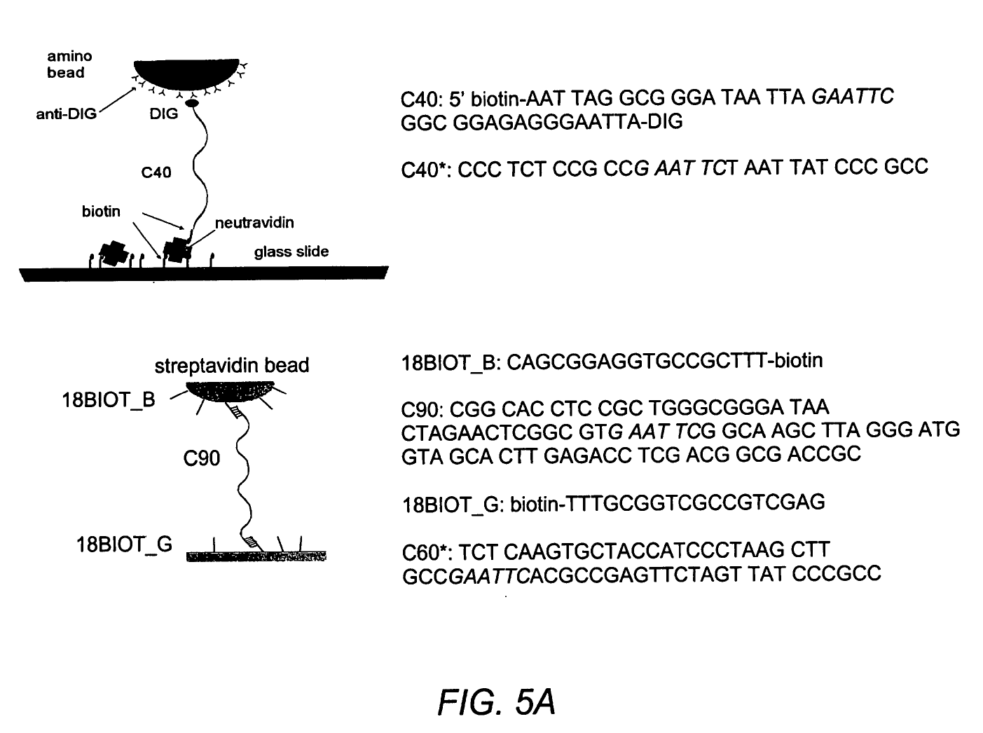

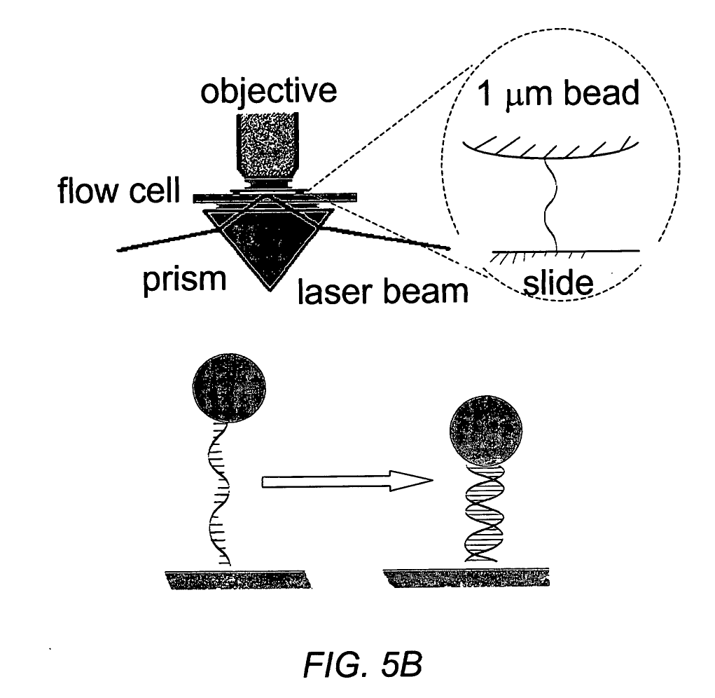

In scheme II, the 93 mer probe C93 was attached to the bead and slide through adapter oligomers 18BIOT-B (SEQ ID NO: 3) and 18BIOT-G (SEQ ID NO: 5) (FIG. 5). 1 μm diameter polystyrene beads functionalized with streptavidin (Sigma) were incubated with 18BIOT-B (0.1 pmoles/μL in PBS) overnight. The batch was then divided into several aliquots; for multiple tether studies, C93 was added in the ratio of 10³ oligos per bead; for single tether studies, the ratio was 5 oligos per bead, or alternatively a mixture in the ratio 1:100 of C90 (SEQ ID NO: 4) and an unrelated 75 met lacking the part complementary to the adaptor oligo on the slide. Finally beads were blocked with excess biotin.

The surface of the flow cell was functionalized by incubating with the following solutions: biotinilated BSA (Sigma) and BSA (fatty acid free, Sigma) in the ratio 1:100, (BSA)=5 mg/mL, in PBS pH=6, overnight; neutravidin (Pierce) 0.1 mg/mL for >4 hrs. For scheme II, biotinilated adapter oligomer 18BIOT-G was introduced (0.1 pmoles/μL, >4 hrs) after the neutravidin step.

Several checks were performed on various aspects of these constructions. Hybridization properties of the oligomers were checked by gel electrophoresis. The specific coupling of the adapter oligomers to the surfaces was checked by fluorescence microscopy. Specific attachment of the beads through the DNA tethers was checked with control beads lacking the tethers and by cutting off tethered beads using a restriction enzyme.

C. Typical Optical Setup.

The principle of the measurement is to create an evanescent optical wave at the glass-solution interface where the beads are tethered. A bead illuminated by this evanescent wave scatters some light. Because the intensity of the evanescent wave decreases (exponentially) with the distance from the interface, the closer a bead is to the interface, the higher the scattered intensity. Thus measuring the scattered intensity yields a measurement of the distance between the bead and the interface: I=I_{c} exp(−h/δ), where I is the scattered intensity, Ic the intensity at contact, h the separation between the bead and the slide, δ the penetration depth of the evanescent wave (δ=86 nm in our setup). Therefore a displacement of the bead can be measured as: h₂−h₁=Δh=δIn (I₂/I₁) (see, e.g. Prieve et al., Langmuir 6, 396-403 (1990); Prieve et al., Applied Optics 32, 1629-41 (1993); Zocchi et al., Biophys. J. 81, 2946-53 (2001); and Singh-Zocchi et al., PNAS 96, 6711-15 (1999)). The optical setup is simple. The flow cell is optically coupled to a Dove prism through immersion oil (FIG. 5). The beam from a 20 mW He—Ne laser is steered through the prism to create an evanescent wave at the bottom of the flow chamber. Light scattered by a single bead is collected through a microscope objective (100×, NA 1.3, oil immersed, Leitz) and focused on a photodiode mounted on a trinocular tube. The signal is recovered through phase sensitive detection: before entering the prism, the beam is chopped (˜1 kHz) and a portion split into a reference detector. Signal and reference are mixed in a lock-in amplifier (Stanford Research) and the output acquired by a computer.

D. Typical Experimental Procedure.

A suspension of beads in buffer TST100 (Tris 20 mM, NaCl 100 mM, Tween 20 μM, pH=8) is introduced in the flow cell. After ˜1 hr some beads have tethered to the bottom and are visible with evanescent wave illumination. A single bead (which appears as a bright diffraction pattern against a dark background) is brought in the field of view of the photodiode. The vertical fluctuations of the bead are monitored for some time; then the hybridization buffer (TST100 for most experiments) containing, as a control, an unrelated 60 mer at a concentration of 100 nM is introduced; finally the same solution with the added target oligomers is introduced.

### Example 2

### Illustrative Embodiment Using a Scatterer to Tag Probe DNA

The experimental data provided herein constitutes a proof of principle of the disclosed methods. In this illustrative example, we describe an embodiment of the invention where a scatterer is used to tag the probe DNA. Experiments were performed in a flow cell built with a microscope slide and cover slip separated by spacers; the typical dimensions of the chamber were 20×20 mm×100 μm thickness. The microscope slide was coupled through immersion oil to the hypotenuse of a Dove prism, and a 20 mW unfocussed He—Ne laser beam was steered through the prism in order to create an evanescent wave (penetration depth Δ=86 nm) at the surface of the slide (the bottom of the cell). In a first set of experiments, a 30 bp oligonucleotide modified with biotin at both ends was coupled to glass beads of approximately 3 μm diameter through a sparse surface concentration of Avidin adsorbed on the beads; prior to coupling the DNA, the beads' free surface was blocked with BSA. The microscope slide was similarly functionalized with Avidin and blocked with BSA. A dilute suspension of the beads was then introduced in the flow cell and the beads were allowed to attach to the bottom of the cell through (multiple) DNA tethers. The light scattered by a single bead was collected through a microscope objective and focused on a photodiode; the intensity was measured through a lock-in detection scheme. Changes in scattered intensity were then converted to changes in the bead's vertical position (the direction normal to the slide) according to:

I=Ic exp(−h/D),

where I is the scattered intensity, Ic is the intensity with the bead in contact with the surface, h is the height of the bead above the surface, D (Δ) is the penetration depth of the evanescent wave (see, e.g. H. Jensenius et al, Phys. Rev. Lett. 79, 5030 (1997)).

### Example 3

### Preferred Methods For the Peparation of Tethered Beads

We successfully employed different strategies to tether beads to the slide through a probe oligomer. In the first strategy, a 40 mer (C40, FIG. 5) modified with biotin at one end and digoxigenin (DIG) at the other end was coupled to anti-digoxigenin antibody (anti-DIG) coated 1 μm diameter beads. These beads attached to the neutravidin functionalized surface of the flow cell used for the measurements (FIG. 5).

In a second strategy, one set of 18 met “adaptors”, biotinilated at one end, is coupled to the neutravidin functionalized flow cell; a second set is coupled to streptavidin coated 1 μm diameter beads. The probe is a 90 mer (C90) with a sequence of 18 bases at the two ends which are complementary to the two adaptors (FIG. 5).

We examined the two extreme cases of low (nominally ˜10 molecules/bead) and high (nominally ˜10³ molecules/bead) probe concentration, giving rise to single and multiple tethers, respectively.

The flow cell is placed in an evanescent wave scattering apparatus where the intensity of light scattered by a single bead tethered to the slide which forms the bottom of the cell (FIG. 5) provides a measurement of the bead-slide separation with sub nm resolution (see, e.g. Zocchi et al., Biophys. J. 81, 2946-53 (2001); and Singh-Zocchi et al., PNAS 96, 6711-15 (1999)).

### Example 4

### Preferred Methods For the Detection of Single Hybridization Events

A tethered bead will change its average position with respect to the slide if the contour length of the tether changes. Hybridization of a target to the tether causes a shortening of the tether as the double helix is formed, thus the hybridization event can be detected. The contour length shortening is 0.9 A per base pair, e.g. 5.4 nm for a 60 mer. Experiments were conducted as follows. The vertical fluctuations of a tethered bead were monitored for a few minutes; then the solution in the flow cell was exchanged for a control consisting of unrelated oligos (60 mers at a concentration of 100 nM). After some time, the solution was exchanged for the same control with added target oligos.

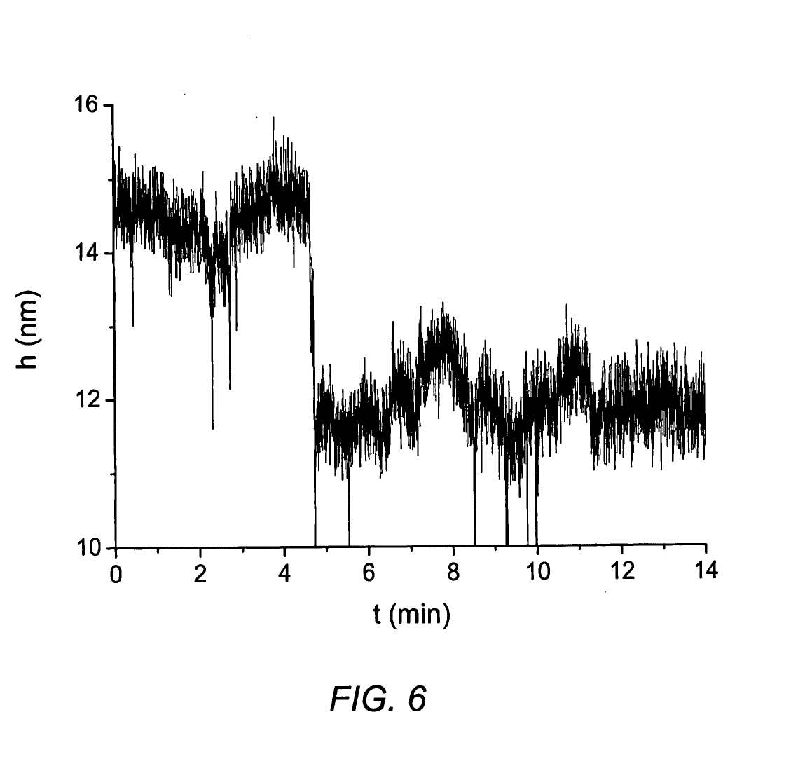

FIG. 6 shows a case where the tether is a 40 mer (C40) and the target a complementary 30 mer. The figure shows the vertical position of a single bead in the course of time. At t≈4.8 min a hybridization event occurs, which pulls the bead towards the surface by ˜2 nm. Thereafter the bead remains in this state.

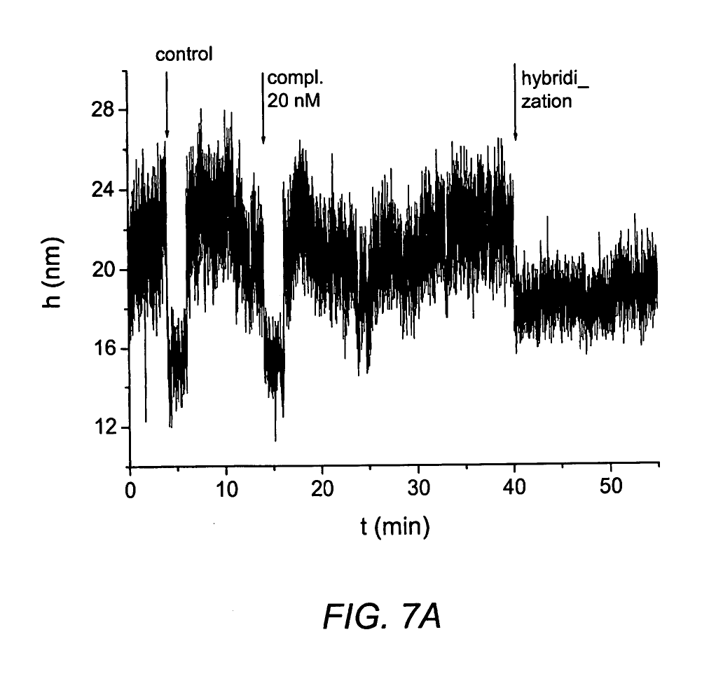

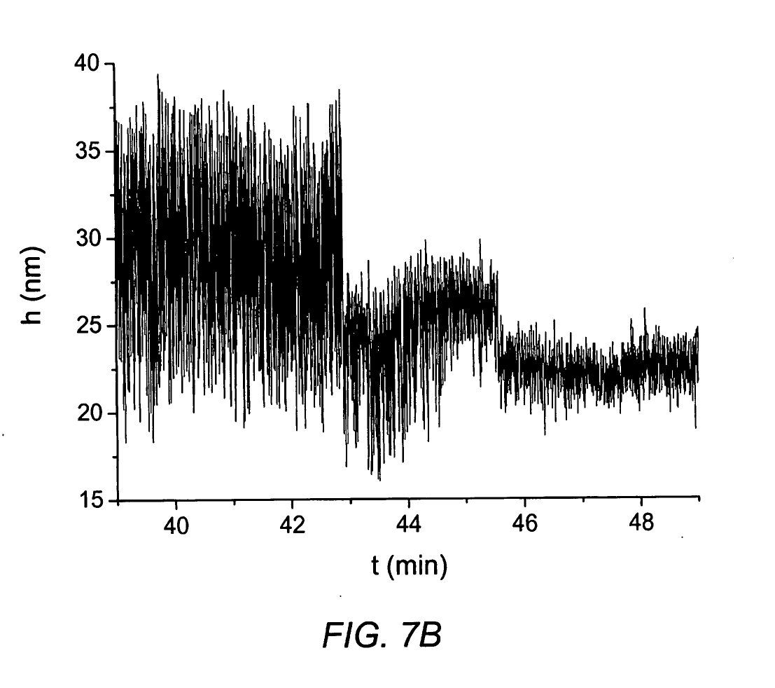

FIG. 7 demonstrates the detection of single hybridization events for decreasing concentration of target, 20 nM and 2 nM. Here the probe is a 90 mer (C90) and the target a 60 mer (C60*) (SEQ ID NO: 6). When a target hybridizes to the tether holding a bead, the bead is pulled towards the surface and its vertical fluctuations are reduced, because excursions away from the surface are more constrained by the reduced contour length of the tether. The magnitude of the effect remains the same independent of target concentration, confirming that we are observing single hybridization events. Consistent with the single molecule picture, these events are always abrupt within our time resolution. This is therefore a direct measurement of the conformational change of a single probe molecule upon hybridization.

### Example 5

### Embodiments of the Invention Having Multiple Tethers

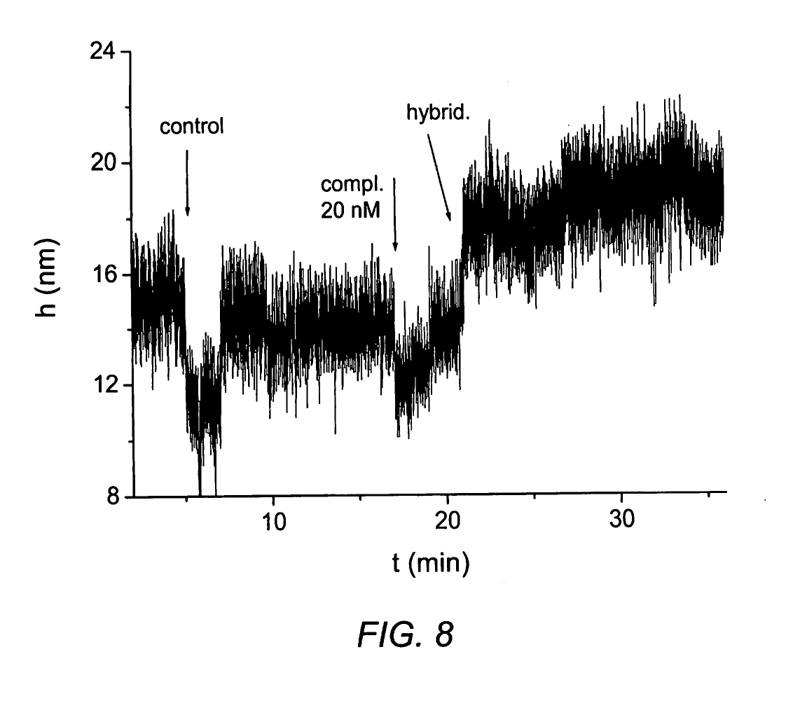

In one embodiment of the invention we explored the opposite extreme case of beads tethered by many probe oligos (beads were prepared with nominally ˜10³ oligos/bead). In this case we observe that in the final state after hybridization the bead is always pushed away from the surface compared to the initial state; an example is shown in FIG. 8. We attribute this effect to the stiffening of the tethers upon hybridization: flexible single strand tethers which are constrained by the random geometry of many attachments into bent states straighten upon hybridization, possibly breaking off some of the more constraining connections and lifting the bead off the surface. In light of the opposite behavior of the two limiting cases, single tether/many tethers, intermediate cases corresponding to more than one but not too many tethers could lead to the two effects fortuitously canceling.

All hybridization assays presently in use employ a relatively large number of probe molecules, e.g. typically 10¹² in the reaction volume of an assay based on beacons. Since the signal increases with the number of hybridized probes, a sufficient number of probes must be hybridized in order to be detectable; for example for the beacons, this is of order 1%. In the present experiment the entire signal comes from the hybridization of a single probe, and is therefore independent of the total amount or concentration of target Thus in principle the method can detect the presence of one single target molecule. There is still a limitation in the minimum concentration, which is practical in terms of the on rate of hybridization. However, in a microfluidic environment, where relevant volumes are of order ˜1 nL, the ˜1 nM target concentration used here corresponds to a total amount of 10⁻¹⁸ moles of target DNA which should be detectable without labeling and without amplification steps.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method of detecting hybridization between a polynucleotide probe and a target polynucleotide having a nucleic acid sequence that is complementary to a nucleic acid sequence in the polynucleotide probe, wherein a first end of the polynucleotide probe is coupled to a matrix and a second end of the polynucleotide probe is coupled to a detectable marker, the method comprising observing a change in the conformation of the polynucleotide probe that is the result of hybridization between the polynucleotide probe and the target polynucleotide.

2. The method of claim 1, wherein the change in the conformation of the polynucleotide probe is observed by observing a change in the height of the detectable marker above the surface of the matrix that results from the hybridization between the polynucleotide probe and the target polynucleotide.

3. The method of claim 1, wherein the change in the conformation of the polynucleotide probe is observed by observing a stiffening of the probe that is the result of hybridization between the polynucleotide probe and the target polynucleotide.

4. The method of claim 2, wherein the change in the height of the detectable marker above the surface of the matrix is observed by evanescent wave scattering.

5. The method of claim 1, wherein the change in the conformation is correlated to the degree of complementarity between the polynucleotide probe and the target polynucleotide.

6. The method of claim 1, wherein the change in the conformation is correlated to the relative amounts of the polynucleotide probe and the target polynucleotide.

7. The method of claim 1, further comprising labelling the target polynucleotide with a detectable marker.

8. The method of claim 1, wherein the polynucleotide probe is about 30 to about 300 nucleotide residues in length.

9. The method of claim 1, wherein the matrix is a gene chip comprising a plurality of polynucleotide probes.

10. The method of claim 1, wherein the detectable marker is a fluorescent compound, a polymer bead or a light scattering particle.

11. The method of claim 1, further comprising creating a negative charge on the surface of the matrix by immobilizing negatively charged molecules on the surface of the matrix.

12. A method of detecting hybridization between a polynucleotide probe and a target polynucleotide having a nucleic acid sequence that is complementary to a nucleic acid sequence in the polynucleotide probe, wherein the polynucleotide probe has a first end labeled with a detectable marker and a second end attached to a matrix having a negative charge, the method comprising using evanescent wave illumination to observe a reduction in the height of a detectable marker coupled to the polynucleotide probe's free end above the surface of the matrix to which the polynucleotide probe is attached.

13. The method of claim 12, wherein the detectable marker is a fluorescent compound or a light scattering particle.

14. The method of claim 12, wherein the target polynucleotide is not labelled with a detectable marker.

15. The method of claim 12, wherein the matrix is a gene chip comprising a plurality of polynucleotide probes.

16. A method of detecting hybridization between a polynucleotide probe and a target polynucleotide having a nucleic acid sequence that is complementary to a nucleic acid sequence in the polynucleotide probe, wherein the polynucleotide probe has a bound end coupled to a matrix and a free end coupled to a detectable marker, the method comprising:
(a) determining an height of the detectable marker coupled to the polynucleotide probe's free end above the surface of the matrix to which the probe is attached in the absence of a complementary polynucleotide sequence;
(b) allowing the polynucleotide probe and the target polynucleotide sequence to come into contact with one another under conditions favorable to hybridization;
(c) using evanescent wave illumination to measure the height of the detectable marker coupled to the polynucleotide probe's free end above the surface of the matrix to which the probe is attached in the presence of the target polynucleotide sequence;
(d) comparing the height of the detectable marker in step (a) with the height of the detectable marker in step (c);
wherein a reduction the height of the detectable marker in step (a) as compared to step (d) is indicative of hybridization between a polynucleotide probe and a target polynucleotide having a nucleic acid sequence that is complementary to a nucleic acid sequence in the polynucleotide probe.

17. An apparatus for detecting hybridization between a polynucleotide probe and a target polynucleotide having a nucleic acid sequence that is complementary to a nucleic acid sequence in the polynucleotide probe, wherein the hybridization is detected using evanescent wave illumination, the apparatus comprising:
(a) a matrix on which a first end of a polynucleotide probe attached, wherein the second end of the polynucleotide probe is coupled to a detectable marker consisting of a fluorophore or a light scattering marker;
(b) a coupling mechanism which optically couples the probe to an optical guide to obtain an evanescent wave on the surface of the matrix;
(c) an optical arrangement which measures the fluorescent or scattered intensity both before and after depositing a solution containing a target polynucleotide sequences on the probe under conditions which favor hybridization of the probe and a target polynucleotide sequences that are complementary to a nucleic acid sequence in the polynucleotide probe; and
(d) a detector which records the difference of fluorescent intensity or scattering before and after subjecting the probe DNA to the target polynucleotide sequences.

18. A kit comprising a container, a label on said container, and a polynucleotide probe composition contained within said container; wherein a first end of the polynucleotide probe is coupled to a matrix and a second end of the polynucleotide probe is coupled to a detectable marker; and instructions for using the polynucleotide probe composition in methods of detecting hybridization between a polynucleotide probe and a target polynucleotide having a nucleic acid sequence that is complementary to a nucleic acid sequence in the polynucleotide probe by observing a change in the conformation of the polynucleotide probe that is the result of hybridization between the polynucleotide probe and the target polynucleotide.

19. The kit of claim 18, wherein the detectable marker is selected to be compatible for use with evanescent wave illumination.

20. The kit of claim 19, wherein the matrix is a gene chip and further wherein the surface of the gene chip is negatively charged.
